(19) United States
(12) Patent Application Publication
(10) Pub. No.: US 2019/0307653 A1
(43) Pub. Date: Oct. 10, 2019

(54) Two-Phase Visual Stimulation and Aromatherapy Bath Bomb Composition

(71) Applicant: Lagunamoon Beauty Limited, Hong Kong (HK)

(72) Inventor: Jianzhong Yang, Guangzhou (CN)

(21) Appl. No.: 15/945,201

(22) Filed: Apr. 4, 2018

(51) Int. Cl. A61K 8/02; A61K 8/19; A61K 8/365; A61K 8/73; A61Q 19/10; A61Q 13/00

(52) U.S. Cl. CPC A61K 8/0233; A61K 8/19; A61Q 13/00; A61K 8/733; A61Q 19/10; A61K 8/365

(57) ABSTRACT

A two-phase visual stimulation and aromatherapy bath bomb composition creates two different phases in a user's visual and aromatherapy bathing experience through an outer layer consisting of sodium bicarbonate, citric acid, tartaric acid, algin, and a surfactant that effervesces when contacting water; and an inner layer consisting of sodium carbonate, citric acid, tartaric acid, an algin that effervesces when contacting water. The inner layer effervesces more rapidly than the outer layer, so as to create two different phases in a user's visual and aromatherapy bathing experience. Thus, as the outer layer effervesces in water in the shower or bath tub, the inner layer, which has at least one different property, indicates to the user that a change in the bath bomb composition has occurred.

### FIELD OF THE INVENTION

The present invention relates generally to a two-phase visual stimulation and aromatherapy bath bomb composition. More so, the present invention relates to a bath bomb composition defined by an outer layer consisting of sodium bicarbonate, citric acid, tartaric acid, algin, and a surfactant that effervesces when contacting water; and an inner layer consisting of sodium carbonate, citric acid, tartaric acid, and an algin that effervesces when contacting water; whereby the inner layer effervesces more rapidly than the outer layer, so as to create two different phases in a user's visual and aromatherapy bathing experience.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Generally, personal care compositions, such as bath foaming and aromatherapy bath compositions exhibit good cleaning properties, good lathering characteristics, and are mild to the skin, so as not to cause drying or irritation. Further, a conditioning benefit to the skin is desirable. Moreover, a pleasant scent is a characteristic of personal care bath compositions.

One such bath care composition is a bath bomb. A bath bomb is a hard-packed mixture of dry ingredients which effervesce when wet. Bath bombs are used to add essential oils, fizz, scent, bubbles, or color to bathwater. The bath bombs' primary ingredients are a weak acid and a bicarbonate base. These are unreactive when dry, but react vigorously when dissolved in water to produce their characteristic fizzing over a period of several minutes. This is an acid-base reaction that involves conversion of citric acid and sodium bicarbonate to sodium citrate and carbon dioxide. Bath bombs also contain scented ingredients as well as dye to impart a pleasant fragrance and color to bathwater. Lathering agents, such as sodium lauryl sulfate, are also often added to create bubble foams.

Other proposals have involved bath bomb compositions. The problem with these bath bomb compositions is that they do not effervesce in two phases. Also, they do not combine an inner layer that effervesces slower than an outer layer, with each layer having different properties and dissolve rates. Even though the above cited bath bomb compositions meets some of the needs of the market, a bath bomb composition defined by an outer layer consisting of sodium bicarbonate, citric acid, tartaric acid, algin, and a surfactant that effervesces when contacting water; and an inner layer consisting of sodium carbonate, citric acid, tartaric acid, and an algin that effervesces when contacting water; whereby the inner layer effervesces more rapidly than the outer layer, is still desired.

### SUMMARY

Illustrative embodiments of the disclosure are generally directed to a two-phase visual stimulation and aromatherapy bath bomb composition. The bath bomb composition serves to create two different phases in a user's visual and aromatherapy bathing experience through an outer layer consisting of sodium bicarbonate, citric acid, tartaric acid, algin, and a surfactant that effervesces when contacting water; and an inner layer consisting of sodium carbonate, citric acid, tartaric acid, an algin that effervesces when contacting water. The inner layer effervesces more rapidly than the outer layer, so as to create two different phases in a user's visual and aromatherapy bathing experience. Thus, as the outer layer effervesces in water in the shower or bath tub, the inner layer, which has at least one different property, indicates to the user that a change in the bath bomb composition has occurred.

In one aspect, a two-phase visual stimulation and aromatherapy bath bomb composition, comprises:
an outer layer consisting of sodium bicarbonate, citric acid, tartaric acid, algin, and a surfactant, the outer layer effervescing when contacting a liquid; and
an inner layer consisting of sodium carbonate, citric acid, tartaric acid, and algin, the inner layer effervescing when contacting the liquid, the inner layer effervescing more rapidly than the outer layer.

In another aspect, the sodium bicarbonate of the outer layer has a percent value approximately between 35% to 65%.

In another aspect, the citric acid of the outer layer has a percent value approximately between 5% to 55%.

In another aspect, the tartaric acid of the outer layer has a percent value approximately between 5% to 55%.

In another aspect, the surfactant has a percent value approximately between 0.1% to 4%.

In another aspect, the alginate comprises sodium alginate.

In another aspect, the sodium carbonate of the inner layer has a percent value approximately between 20% to 65%.

In another aspect, the citric acid of the inner layer has a percent value approximately between 5% to 55%.

In another aspect, the tartaric acid of the inner layer has a percent value approximately between 5% to 55%.

In yet another aspect, the liquid is water.

In another aspect, the composition comprises a capsule.

In another aspect, the capsule has a generally disc-shape.

In another aspect, the outer layer is larger than the inner layer.

In yet another aspect, the inner layer has at least one different property than the outer layer.

In another aspect, the composition comprises a scent, or a dye, or both.

One objective of the present invention is to add essential oils, scent, bubbles or color to bathwater.

Another objective is to impart a pleasant fragrance and color to bathwater.

Yet another objective is to provide a therapeutic fizzing in the bathwater.

Yet another objective is to create two different phases in a user's visual and aromatherapy bathing experience.

Yet another objective is to indicate to the user that a change in the bath bomb composition has occurred.

Yet another objective is to provide a bath bomb composition that creates a pleasant smell and conditions the skin when introduced into bathwater.

Yet another objective is to provide an inexpensive to manufacture bath bomb.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a Table, showing the ingredients for the outer and inner layers of the bath bomb composition, in accordance with an embodiment of the present invention; and

FIG. 2 illustrates a sectioned top view of an exemplary capsule-shaped bath bomb composition, showing the outer and inner layers, in accordance with an embodiment of the present invention.

Like reference numerals refer to like parts throughout the various views of the drawings.

### DETAILED DESCRIPTION OF THE INVENTION

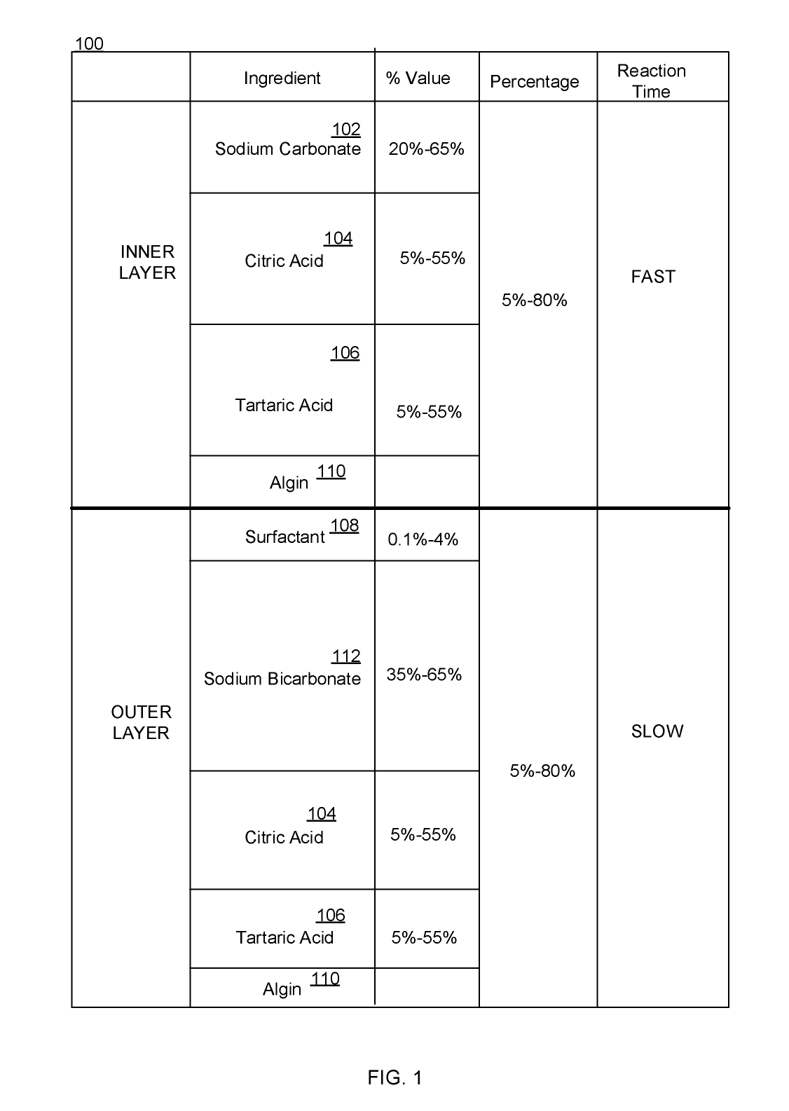

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

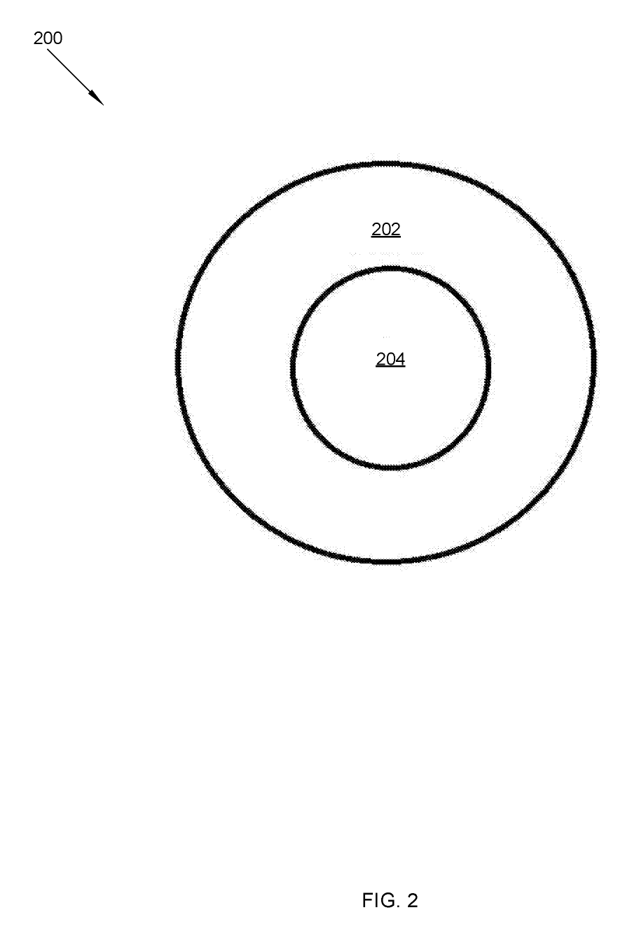

A two-phase visual stimulation and aromatherapy bath bomb composition 100 is referenced in FIGS. 1-2. The bath bomb composition 100 provides an invigorating multilayered explosion of bubbles, color, and scent in a bath or shower through use of separate layers of ingredients that effervesce at different rates to create two different phases in a user's visual and aromatherapy bathing experience. The ingredients from each layer effervesce at different rates, so that the escape of gas creates a tantalizing foaming and fizzing sensation in the bathwater.

In some embodiments, the bath bomb composition 100 comprises an outer layer 202 and an inner layer 204, with each layer having different properties and rates of effervescence. The bath bomb composition 100 may include a capsule 200 that is disc-shaped, with the outer layer 202 fully encapsulating the inner layer 204. Thus, as the outer layer 202 dissolves, the inner layer 204 commences the effervesce dissolving. For example, as the outer layer 202 effervesces in water in a bathtub, the inner layer 204, which has at least one different property than the outer layer 202, indicates to the user that a change in the bath bomb composition 100 has occurred.

As the Table in FIG. 1 illustrates, the outer layer 202 of the bath bomb composition 100 consists of sodium bicarbonate 112, citric acid 104, tartaric acid 106, algin 110, and a surfactant 108 that effervesces when contacting a liquid. In one non-limiting embodiment, the sodium bicarbonate 112 of the outer layer 202 has a percent value approximately between 35% to 65%. The citric acid 104 of the outer layer 202 has a percent value approximately between 5% to 55%. The tartaric acid 106 of the outer layer 202 has a percent value approximately between 5% to 55%. The surfactant 108 of the outer layer 202 has a percent value approximately between 0.1% to 4%. The algin 110 may include a sodium alginate. In another embodiment, the overall percentage of the outer layer 202 is about between 5% to 80%.

The interaction between the outer layer 202 and the liquid forms an acid-base reaction that involves conversion of citric acid 104 and sodium bicarbonate 112 to sodium citrate and carbon dioxide. C₅H₇O₅CO₂H_{(aq.)}+NaHCO_{3 (aq.)}→C₅H₇O₅CO₂⁻Na⁺_{(aq.)}+H₂O₍₁₎+CO_{2 (g)}. This reaction creates effervescence and releases gases, scents, and oils into the bathwater. A similar reaction occurs with the inner layer 204, as discussed below.

As discussed above, the inner layer 204 effervesces more rapidly than the outer layer 202, so as to create two different phases in a user's visual and aromatherapy bathing experience. The inner layer 204 consists of sodium carbonate 102, citric acid 104, tartaric acid 106, an algin 110 that effervesces when contacting the liquid. The liquid may include, without limitation, water, such as bathwater.

In one non-limiting embodiment, the sodium carbonate 102 of the inner layer 204 has a percent value approximately between 20% to 65%. The citric acid 104 of the inner layer 204 has a percent value approximately between 5% to 55%. The tartaric acid 106 of the inner layer 204 has a percent value approximately between 5% to 55%. In another embodiment, the overall percentage of the outer layer 202 is about between 5% to 80%.

As FIG. 2 illustrates, the composition 100 can include a disc-shaped capsule 200. Though in other embodiments, a disc-shaped capsule is not used; but rather, different shape for the capsule 200 may be used. In yet another embodiment, the outer layer 202 is larger than the inner layer 204. And because the outer and inner layers 202, 204 have different ingredients—even if slightly different—the inner layer 204 has at least one different property than the outer layer 202. This is what creates two different phases in a user's visual and aromatherapy bathing experience. The ingredients from each layer 202, 204 effervesce at different rates, so that the escape of gas creates a tantalizing foaming and fizzing sensation in the bathwater.

In some embodiments the composition 100 may further include a scent, a dye, a lathering agent, or combinations thereof. These additional ingredients can be added to both, or just one of the layers 202, 204. For example, a vanilla scent and a cream coloring could be added to the outer layer 202, and a rose fragrance with a red dye could be added to the inner layer 204. These ingredients, known in the art of bath products, further enhance the visual therapeutic and aromatherapy effects of the bath bomb composition 100.

In use, the bathtub is filled with water. The capsule 200 of bath bomb composition 100 is placed in the bathwater. The outer layer 202 effervesces to release the ingredients as a gas that mixes with the bathwater. Various oils, scents, and dyes are released into the bathwater in multiple phases. The outer layer 202, which is larger than the inner layer 204, effervesces at a slower rate than the inner layer 204. As the outer layer 202 finishes, the inner layer 204 is exposed. The inner layer 204 then begins effervescing. The user will notice a change in the effect of the bath bomb composition 100 between the two layers 202, 204. This creates two different phases in a user's visual and aromatherapy bathing experience.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A two-phase visual stimulation and aromatherapy bath bomb composition, the composition consisting of:
an outer layer consisting of sodium bicarbonate, citric acid, tartaric acid, algin, and a surfactant, the outer layer effervescing when contacting a liquid; and
an inner layer consisting of sodium carbonate, citric acid, tartaric acid, and algin, the inner layer effervescing when contacting the liquid, the inner layer effervescing more rapidly than the outer layer.

2. The composition of claim 1, wherein the sodium bicarbonate of the outer layer has a percent value approximately between 35 percent to 65 percent.

3. The composition of claim 1, wherein the citric acid of the outer layer has a percent value approximately between 5 percent to 55 percent.

4. The composition of claim 1, wherein the tartaric acid of the outer layer has a percent value approximately between 5 percent to 55 percent.

5. The composition of claim 1, wherein the surfactant has a percent value approximately between 0.1 percent to 4 percent.

6. The composition of claim 1, wherein the sodium carbonate of the inner layer has a percent value approximately between 20 percent to 65 percent.

7. The composition of claim 1, wherein the alginate comprises sodium alginate.

8. The composition of claim 1, wherein the liquid is water.

9. The composition of claim 1, wherein the composition comprises a capsule.

10. The composition of claim 9, wherein the capsule has a generally disc-shape.

11. The composition of claim 1, wherein the outer layer is larger than the inner layer.

12. The composition of claim 1, further comprising a scent, or a dye, or both.

13. A two-phase visual stimulation and aromatherapy bath bomb composition, the composition consisting of:
an outer layer consisting of sodium bicarbonate having a percent value approximately between 35 percent to 65 percent, citric acid having a percent value approximately between 5 percent to 55 percent, tartaric acid having a percent value approximately between 5 percent to 55 percent, algin, and a surfactant having a percent value approximately between 0.1 percent to 4 percent, the outer layer effervescing when contacting water; and
an inner layer consisting of sodium carbonate having a percent value approximately between 20 percent to 65 percent, citric acid having a percent value approximately between 5 percent to 55 percent, tartaric acid having a percent value approximately between 5 percent to 55 percent, and algin,
whereby the inner layer effervesces when contacting the water,
whereby the inner layer effervesces more rapidly than the outer layer.

14. The composition of claim 13, wherein the alginate comprises sodium alginate.

15. The composition of claim 13, wherein the composition comprises a capsule.

16. The composition of claim 15, wherein the capsule has a generally disc-shape.

17. The composition of claim 13, wherein the outer layer is larger than the inner layer.

18. The composition of claim 13, wherein the inner layer has at least one different property than the outer layer.

19. A two-phase visual stimulation and aromatherapy bath bomb composition, the composition consisting of:
an outer layer consisting of sodium bicarbonate having a percent value approximately between 35 percent to 65 percent by weight of the composition, citric acid having a percent value approximately between 5 percent to 55 percent by weight of the composition, tartaric acid having a percent value approximately between 5 percent to 55 percent by weight of the composition, algin, and a surfactant having a percent value approximately between 0.1% to 4% by weight of the composition, the outer layer effervescing when contacting water; and
an inner layer consisting of sodium carbonate having a percent value approximately between 20 percent to 65 percent by weight of the composition, citric acid having a percent value approximately between 5 percent to 55 percent by weight of the composition, tartaric acid having a percent value approximately between 5 percent to 55 percent by weight of the composition, and algin,
whereby the inner layer effervesces when contacting the water,
whereby the inner layer effervesces more rapidly than the outer layer.

20. The composition of claim 19, wherein the outer layer is larger than the inner layer.
